(19) United States
(12) Patent Application Publication
(10) Pub. No.: US 2015/0342528 A1
(43) Pub. Date: Dec. 3, 2015

(54) ELECTRONIC APPARATUS AND VITAL SIGN SIGNAL MEASURING METHOD

(71) Applicant: KABUSHIKI KAISHA TOSHIBA, Tokyo (JP)

(72) Inventors: Takashi Sudo, Fuchu-shi (JP); Yasuhiro Kanishima, Tokyo (JP)

(21) Appl. No.: 14/823,778

(22) Filed: Aug. 11, 2015

Related U.S. Application Data
(63) Continuation of application No. PCT/JP2013/063421, filed on May 14, 2013.

(51) Int. Cl. A61B 5/00; A61B 5/044; A61B 5/0408; A61B 5/024

(52) U.S. Cl. CPC A61B 5/6843; A61B 5/02438; A61B 5/4884; A61B 5/7475; A61B 5/6897; A61B 5/044; A61B 5/04085; A61B 5/0006; A61B 2560/0468; A61B 2562/0209

(57) ABSTRACT

According to one embodiment, an electronic apparatus determines whether a vital sign sensor is in contact with a human body and determines whether a contact state between the vital sign sensor and the human body is stable. The apparatus obtains an effective time-series signal by removing, from an output time-series signal of the vital sign sensor, a first time-series signal and a second time-series signal, the first time-series signal corresponding to a time period of a non-contact state and a second time-series signal corresponding to a time period of an unstable state. The apparatus analyzes the effective time-series signal to measure a value associated with a vital sign signal of the human body.

### CROSS REFERENCE TO RELATED APPLICATIONS

This application is a Continuation Application of PCT Application No. PCT/JP2013/063421, filed May 14, 2013, the entire contents of which are incorporated herein by reference.

### FIELD

Embodiments described herein relate generally to a technique for processing vital sign signals.

### BACKGROUND

Recently, home-based preventive medical care and health care have been attracting attention. At the same time, reduction in the size of medical equipment has been progressing.

In general, however, dedicated apparatuses are necessary to measure vital sign signals such as a pulse wave or an electrocardiogram.

Further, development of techniques for measuring the pulse wave with a home-use electronic apparatus such as an optical mouse has started recently.

In general, the user is required to remain motionless for a long time with a sensor portion of an apparatus in contact with the body during measurement of a vital sign signal. Thus, realization of a new technique allowing a vital sign signal from the user to be easily measured while the user operates a general home-use electronic apparatus such as a personal computer is required.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary perspective view showing an outer appearance of an electronic apparatus according to an embodiment which comprises a palm rest area in which two electrocardiographic electrodes and a pulse wave sensor are arranged.

FIG. 2 is an exemplary perspective view showing an outer appearance of the electronic apparatus according to the embodiment which comprises a palm rest area in which two electrocardiographic electrode plates and a pulse wave sensor arranged in an opening in one of the two electrocardiographic electrode plates are arranged.

FIG. 3 is an exemplary perspective view showing an outer appearance of a mouse configured to communicate with the electronic apparatus according to the embodiment.

FIG. 4 is an exemplary perspective view showing an outer appearance of a remote-control unit configured to communicate with the electronic apparatus according to the embodiment.

FIG. 5 is an exemplary block diagram showing a system configuration of the electronic apparatus according to the embodiment.

FIG. 6 is an exemplary block diagram showing a relationship between a measurement engine provided in the electronic apparatus according to the embodiment and a component around the measurement engine.

FIG. 7 is an exemplary view for illustrating an operation for removing a signal portion in a time period other than that of a stable state from a detection signal of a vital sign sensor, the operation being performed by the electronic apparatus according to the embodiment.

FIG. 8 is an exemplary view for illustrating a frequency characteristic of an electrocardiogram signal portion corresponding to a time period of a non-contact state, the time period being detected by the electronic apparatus according to the embodiment.

FIG. 9 is an exemplary view for illustrating a frequency characteristic of an electrocardiogram signal portion corresponding to a time period in which a hand moves, the time period being detected by the electronic apparatus according to the embodiment.

FIG. 10 is an exemplary view for illustrating a frequency characteristic of an electrocardiogram signal portion corresponding to a time period in which a contact state is unstable, the time period being detected by the electronic apparatus according to the embodiment.

FIG. 11 is an exemplary view for illustrating a frequency characteristic of an electrocardiogram signal portion corresponding to a time period in which a contact state is stable, the time period being detected by the electronic apparatus according to the embodiment.

FIG. 12 is an exemplary block diagram for illustrating processing of a pulse wave signal which is executed by the electronic apparatus according to the embodiment.

FIG. 13 is an exemplary view for illustrating a frequency characteristic of a pulse wave signal portion corresponding to a time period of a non-contact state, the time period being detected by the electronic apparatus according to the embodiment.

FIG. 14 is an exemplary view for illustrating a frequency characteristic of a pulse wave signal portion corresponding to a time period in which a hand moves, the time period being detected by the electronic apparatus according to the embodiment.

FIG. 15 is an exemplary view for illustrating a frequency characteristic of a pulse wave signal portion corresponding to a time period in which a contact state is unstable, the time period being detected by the electronic apparatus according to the embodiment.

FIG. 16 is an exemplary view for illustrating a stress level (stress index) calculating operation executed by the electronic apparatus according to the embodiment.

FIG. 17 is an exemplary view for illustrating a measurement result concerning a pulse, a blood pressure, and stress presented to the user by the electronic apparatus according to the embodiment.

FIG. 18 is an exemplary view for illustrating a measurement result concerning stress presented to the user by the electronic apparatus according to the embodiment.

FIG. 19 is an exemplary block diagram for illustrating a collaborative operation of the electronic apparatus according to the embodiment and a mouse.

FIG. 20 is an exemplary flowchart for illustrating a procedure of measuring processing executed by the electronic apparatus according to the embodiment.

### DETAILED DESCRIPTION

Various embodiments will be described hereinafter with reference to the accompanying drawings.

In general, according to one embodiment, an electronic apparatus includes a determination controller and a measurement controller. The determination controller determines whether a vital sign sensor is in contact with a human body and determines whether a contact state between the vital sign sensor and the human body is stable. The measurement controller obtains an effective time-series signal by removing, from an output time-series signal of the vital sign sensor, a first time-series signal and a second time-series signal, the first time-series signal corresponding to a time period of a non-contact state where the vital sign sensor is not in contact with the human body and a second time-series signal corresponding to a time period of an unstable state where a contact state between the vital sign sensor and the human body is unstable. The measurement controller analyzes the effective time-series signal to measure a value associated with a vital sign signal of the human body.

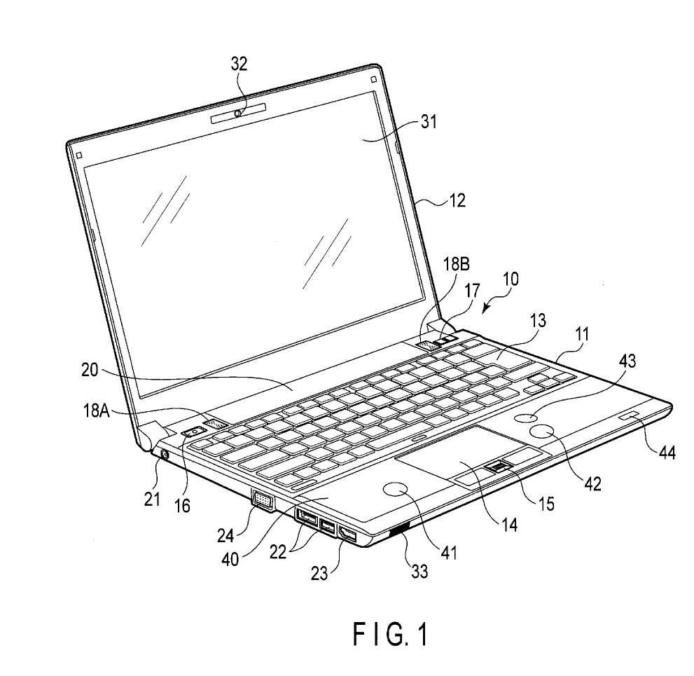

First, referring to FIG. 1, a structure of an electronic apparatus according to an embodiment will be described. This electronic apparatus is configured to execute processing according to an operation of an input device (for example, keyboard, mouse, and remote-control unit) performed by the user. This electronic apparatus is a general home-use electronic apparatus such as a personal computer or a TV. A case where this electronic apparatus is realized as a notebook portable personal computer 10 is hereinafter assumed.

FIG. 1 is a perspective view of the computer 10 with display unit opened, viewed from the front side. The computer 10 is configured to receive power from a battery 20. The computer 10 comprises a computer main body 11 and a display (display unit) 12 attached to the computer main body 11. A display device such as a liquid crystal display (LCD) 31 is embedded in the display unit 12. Furthermore, a camera (web camera) 32 is arranged in the upper end of the display unit 12.

The display unit 12 is attached to the computer main body 11 rotatable between an opened position at which the upper surface of the computer main body 11 is exposed and closed position at which the upper surface of the computer main body 11 is covered with the display unit 12. The computer main body 11 comprises a thin box housing, and a keyboard 13, a touchpad 14, a fingerprint sensor 15, a power switch 16 for powering on/off the computer 10, some function buttons 17, and speakers 18A and 18B are arranged on its upper surface.

Further, the computer main body 11 is provided with a power connector 21. The power connector 21 is provided on a side surface, for example, the left side surface of the computer main body 11. An external power-supply is detachably connected to the power connector 21. An AC adaptor can be used as t external power-supply. The AC adaptor is a power-supply for converting commercial power (AC power) into DC power.

The battery 20 is detachably mounted, for example, in the back end of the computer main body 11. The battery 20 may be a battery built in the computer 10.

The computer 10 is driven by power from an external power-supply or power from the battery 20. If the external power-supply is connected to the power connector 21 of the computer 10, the computer 10 is driven by the power from the external power-supply. Further, the power from the external power-supply is also used to charge the battery 20. The computer 10 is driven by the power from the battery 20 while the external power-supply is not connected to the power connector 21 of the computer 10.

Moreover, the computer main body 11 is provided with some USB ports 22, a High-definition Multimedia Interface (HDMI) output port 23, and an RGB port 24.

Moreover, an infrared light receiving unit 33 for communicating with an external remote-control unit is arranged on the front surface of the computer main body 11. The external remote-control unit is used to remotely control a television (TV) function of the computer 10. The TV function of the computer 10 comprises a function of displaying frames corresponding to video data included in predetermined program data broadcast by a TV broadcast signal on the LCD 31, a function of recording predetermined program data in a storage medium, a function of reproducing the recorded program data, etc.

Moreover, the computer 10 comprises a vital sign sensor configured to detect a vital sign signal such as an electrocardiogram (ECG) and a pulse wave.

In the present embodiment, the vital sign sensor is arranged in an input device, or arranged in a specific portion on the housing of the computer 10 which a hand contacts when the input device is operated, allowing the vital sign signal to be automatically measured while the user operates the computer 10.

In FIG. 1, the vital sign sensor is arranged in a palm rest area 40 on the upper surface of the computer main body 11. A position on the palm rest area 40 at which the vital sign sensor is arranged is a position with which a palm of the user comes into contact when the user puts his fingers of both hands at a home position of the keyboard 13.

In the present embodiment, the computer 10 comprises first and second electrocardiogram (ECG) electrodes 41 and 42, and pulse wave sensor 43 as the above vital sign sensor. A plethysmogram (PG) can be used as pulse wave sensor 43. First and second electrocardiographic electrodes 41 and 42, and pulse wave sensor 43 are arranged on the palm rest area 40 to be exposed.

First and second electrocardiographic electrodes 41 and 42 function as an electrocardiogram sensor for obtaining an electrocardiogram of the user. First and second electrocardiographic electrodes 41 and 42 are arranged to be in contact with skin at two points sandwiching a heart of the user, that is, the left palm and the right palm, respectively. In the present embodiment, first and second electrocardiographic electrodes 41 and 42 are arranged on both sides of the touchpad 14 such that the left palm naturally comes in contact with first electrocardiographic electrode 41, and the right palm naturally comes in contact with second electrocardiographic electrode 42 when the user puts his fingers of both hands at the home position of the keyboard 13. That is, first electrocardiographic electrode 41 is arranged at a position on the palm rest area 40 located on the left side of the touchpad 14, and second electrocardiographic electrode 42 is arranged at a position on the palm rest area 40 located on the right side of the touchpad 14.

Pulse wave sensor 43 is a sensor configured to detect a pulse wave (here, plethysmogram). Pulse wave sensor 43 can be realized by a photopelthysmograph (PPG) sensor. In this case, pulse wave sensor 43 comprises a light-emitting element (for example, green LED) which is a light source, and a photodiode (PD) which is a light receiving unit. Pulse wave sensor 43 irradiates the surface of skin with light through a window portion arranged on the palm rest area 40, and grasps variation of reflected light changed by blood flow change in a capillary vessel through the window portion using the photodiode (PD).

In the present embodiment, pulse wave sensor 43 (PPG sensor) is arranged on the palm rest area 40 in proximity to either first electrocardiographic electrode 41 or second electrocardiographic electrode 42 to allow the measurement of the electrocardiogram and that of the pulse wave to be simultaneously executed. In the example of FIG. 1, pulse wave sensor 43 is arranged on the palm rest area 40 in proximity to second electrocardiographic electrode 42.

The computer 10 analyzes at least one of output time-series signals of the electrocardiogram sensors (electrocardiographic electrodes 41 and 42) and that of pulse wave sensor 43, and measures a value concerning a vital sign signal of the user (human body). The output time-series signals of the electrocardiogram sensors (electrocardiographic electrodes 41 and 42) are a time-series signal obtained by sampling a difference in potential between electrocardiographic electrodes 41 and 42. The output time-series signal of pulse wave sensor 43 is a time-series signal obtained by sampling an output signal of pulse wave sensor 43.

The above-described value concerning the vital sign signal is a value, etc., obtained by digitizing a biological phenomenon. The LCD 31 can display the value concerning the vital sign signal obtained by measurement. The value concerning the vital sign signal displayed on the LCD 31 is, for example, a pulse, a blood pressure, and a stress level.

More specifically, the computer 10 can measure an electrocardiogram, a heart rate/pulse rate, an R-R interval, a stress level, a blood pressure, etc. The electrocardiogram can be obtained by analyzing the output time-series signals of first and second electrocardiographic electrodes 41 and 42. The heart rate can be obtained from the electrocardiogram, and the pulse rate can be calculated by analyzing the output time-series signal of pulse wave sensor 43.

In the measurement of the stress level, pulse interval data indicating variation of a pulse interval is obtained based on the output time-series signal of pulse wave sensor 43. The pulse interval data is time-series data comprising a plurality of sample values, each of which indicates a pulse interval. Then, a power spectrum of a low-frequency domain and that of a high-frequency domain can be obtained by converting the pulse interval data for a predetermined time period into frequency spectrum distribution. Then, the stress level can be measured based on the power spectrum of the low-frequency domain and that of the high-frequency domain.

In the measurement of the blood pressure, a pulse wave transit time (PWTT) is obtained based on a peak of an electrocardiogram waveform (R wave) and that of the pulse wave. The pulse wave transit time indicates a time interval from appearance of the R wave of the electrocardiogram to appearance of a peripheral pulse wave. The pulse wave transit time is inversely proportional to the blood-pressure value. Thus, the variation of the blood pressure can be obtained from the pulse wave transit time (PWTT).

In the measurement of the blood pressure, an initial value may be pre-input in the computer 10. For example, a blood-pressure value of the user measured by an ordinary pressure measurement apparatus, and a pulse wave transit time at that moment may be pre-input in the computer 10 as an initial value. A current blood-pressure value of the user can be obtained using variation of a blood pressure obtained from a current pulse wave transit time (PWTT), and the initial value (relationship between the blood-pressure value and pulse wave transit time).

Alternatively, normal data indicating the relationship between the blood-pressure value and the pulse wave transit time may be prepared to obtain the current blood-pressure value of the user using this normal data and the variation of the blood pressure obtained from the current pulse wave transit time (PWTT), instead of inputting the blood-pressure value of the user measured by an ordinary pressure measurement apparatus and a pulse wave transit time at that moment as initial values.

Moreover, the computer main body 11 comprises an indicator 44. The indicator 44 can function as a state display unit for informing the user that the vital sign signal is being measured. The indicator 44 may be at least one LED. Further, the indicator 44 may present to the user a state indicating whether it is a stable state where the user (human body) is stably in contact with vital sign sensors (electrocardiographic electrodes 41 and 42, and pulse wave sensor 43), etc.

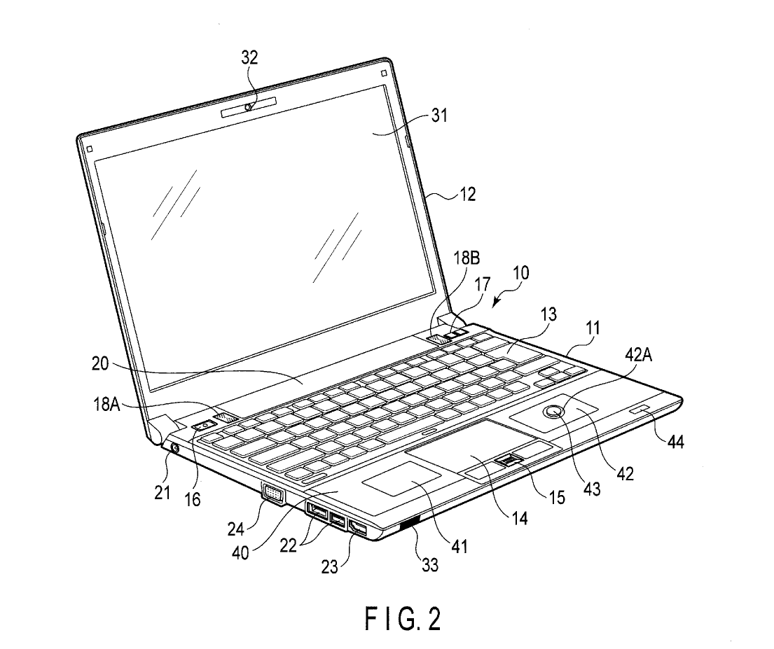

The arrangement of electrocardiographic electrodes 41 and 42 and pulse wave sensor 43 is not limited to the example shown in FIG. 1. For example, as shown in FIG. 2, first and second electrocardiographic electrodes 41 and 42 may be first and second electrocardiographic electrode plates arranged on both sides of the touchpad 14 on the palm rest area 40. As the electrocardiographic electrode plates, a thin metallic plate can be used. The electrocardiographic electrode plate which functions as second electrocardiographic electrode 42 comprises a hollow opening 42A. Pulse wave sensor (PPG sensor) 43 is arranged in the opening 42A to be exposed through the opening 42A provided on an electrocardiographic electrode plate 42. This structure allows a palm to easily come in contact with the electrocardiographic electrode plate 42 and pulse wave sensor (PPG sensor) 43 at the same time.

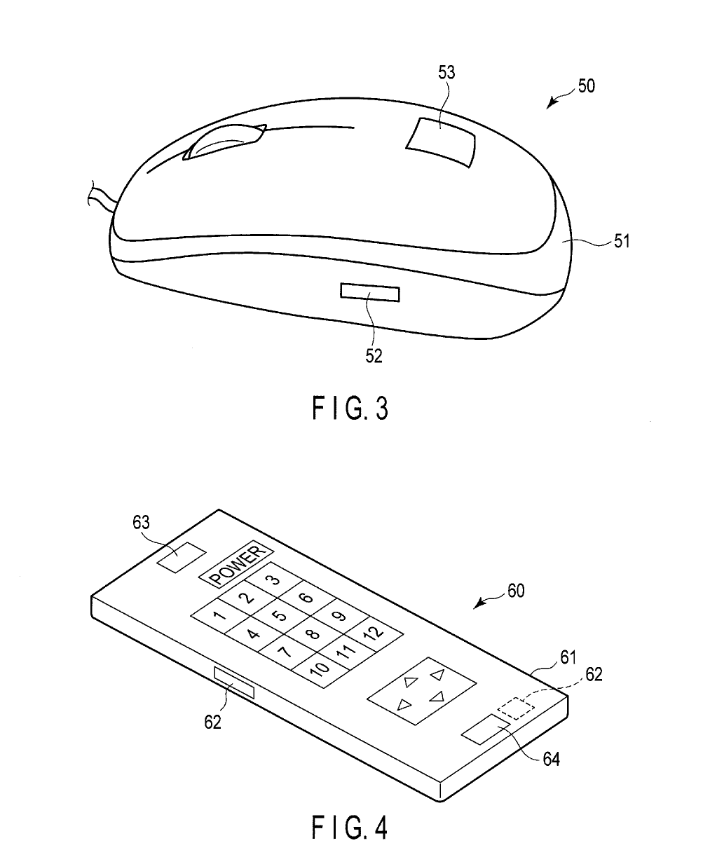

Although the example in which the vital sign sensor is arranged in the palm rest area on the upper surface of the computer main body 11 is described in FIG. 1, the vital sign sensor may be arranged in a mouse 50 configured to communicate with the computer 10, as shown in FIG. 3, in addition to or instead of this.

FIG. 3 shows a mouse 50 for a right-handed person. In the mouse 50, pulse wave sensor 52 is arranged at a position near the central portion of the left side surface of a mouse main body 51 to be exposed such that pulse wave sensor 52 comes in contact with a right thumb when the user operates the mouse 50. Pulse wave sensor 52 may be the PPG sensor. Furthermore, electrocardiographic electrode 53 for a right hand is arranged on part of the upper surface of the mouse main body 51 to be exposed such that a right palm comes in contact with electrocardiographic electrode 53 for the right hand.

An output time-series signal of pulse wave sensor 52 and that of electrocardiographic electrode 53 for the right hand are transmitted to the computer 10 through a cable such as a USB cable, or wirelessly transmitted to the computer 10.

The computer 10 can acquire the output time-series signal of pulse wave sensor 52 from the mouse 50, even if the user operates only the mouse 50 with the right hand without operating the keyboard 13. Further, if the user operates the mouse 50 with the right hand with the left hand of the user put on a home position on the palm rest area 40, the left palm of the user comes in contact with first electrocardiographic electrode 41 on the palm rest area 40, and the right palm comes in contact with electrocardiographic electrode 53 of the mouse 50. Thus, the electrocardiogram can be measured by analyzing the output time-series signal obtained by sampling a difference in potential between electrocardiographic electrodes 41 and 53.

Incidentally, in a mouse for a left-handed person, pulse wave sensor 52 may be arranged at a position near the central portion of the right side surface of the mouse main body 51 to be exposed such that pulse wave sensor 52 comes in contact with a thumb of the left hand when the user operates this mouse main body.

Further, the vital sign sensor may be arranged in a remote-control unit 60 configured to communicate with the computer 10, as shown in FIG. 4, in addition to or instead of arranging the vital sign sensor in the palm rest area 40 on the upper surface of the computer main body 11. The remote-control unit 60 is used to remotely control a TV function (turning on/off TV function, channel switching, etc.) of the computer 10.

As shown in FIG. 4, a plurality of buttons (power button, group of channel switching buttons, arrow key, etc.) for remotely controlling the computer 10 are arranged on the upper surface of a remote-control unit main body 61.

Pulse wave sensor (PPG sensor) 62 is arranged on the left side surface of the remote-control unit main body 61, for example, near the central portion of the left side surface to be exposed. Further, first and second electrocardiographic electrodes 63 and 64 are arranged in the upper end and lower end on the upper surface of the remote-control unit main body 61, respectively.

Further, pulse wave sensor 62 may be arranged in the upper end or lower end on the upper surface of the remote-control unit main body 61 to come close to first electrocardiographic electrode 63 or second electrocardiographic electrode 64. In this case, either first electrocardiographic electrode 63 or second electrocardiographic electrode 64 may be realized by the metallic plate comprising the opening as described in FIG. 2, and pulse wave sensor 63 may be arranged in this opening such that pulse wave sensor 62 is exposed through the opening.

The measurement of the electrocardiogram and that of the pulse wave can be simultaneously performed by causing the user to grasp the upper end of the remote-control unit main body 61 with the left hand, and to grasp the lower end of the remote-control unit main body 61 with the right hand.

The output time-series signal of pulse wave sensor 62 and that corresponding to a difference in potential between two electrocardiographic electrodes 63 and 64 may be transmitted from the remote-control unit 60 to the computer 10 in a wireless communication system different from infrared light, for example, a wireless communication system such as a wireless LAN and Bluetooth (registered trademark).

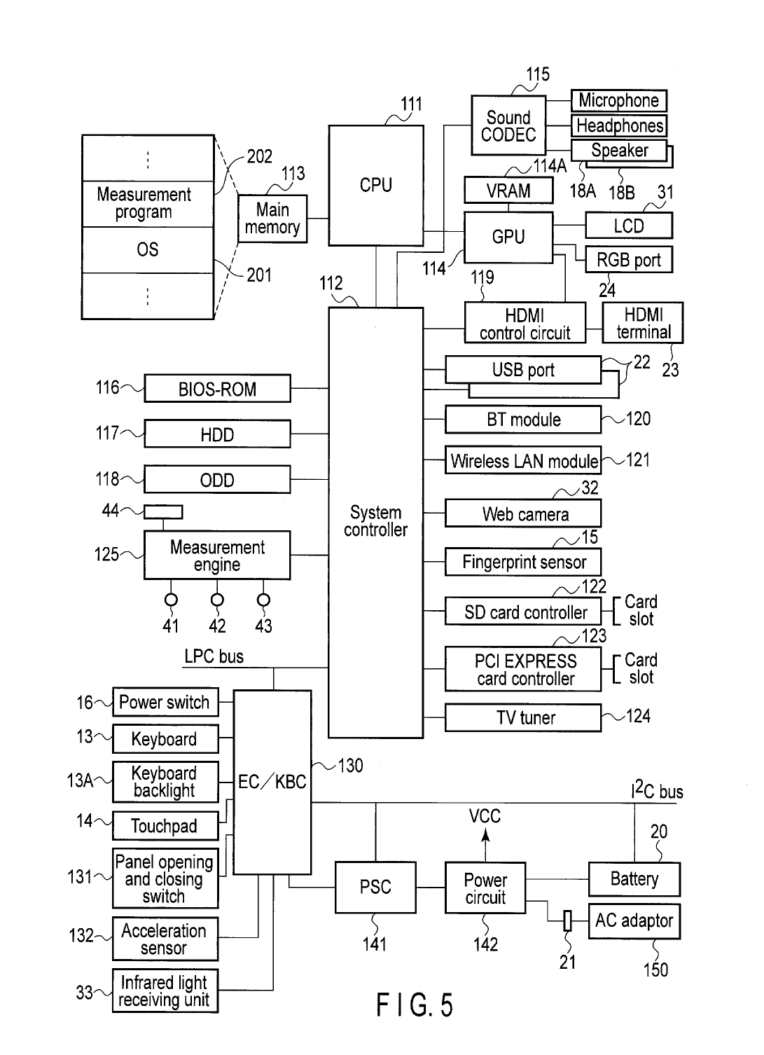

FIG. 5 shows a system configuration of the computer 10. The computer 10 comprises a CPU 111, a system controller 112, the main memory 113, a graphics processing unit (GPU) 114, a sound codec 115, a BIOS-ROM 116, a hard disk drive (HDD) 117, an optical disk drive (ODD) 118, a Bluetooth (registered trademark) module 120, a wireless LAN module 121, an SD card controller 122, a PCI EXPRESS card controller 123, a TV tuner 124, a measurement engine 125, an embedded controller/keyboard controller IC (EC/KBC) 130, a keyboard backlight 13A, a panel opening and closing switch 131, an acceleration sensor 132, a power-supply controller (PSC) 141, a power-supply circuit 142, etc. To prevent the vital sign sensor from being affected by electromagnetism or vibration caused by the HDD 117, a solid state drive (SSD) may be provided instead of the HDD 117.

The CPU 111 is a processor configured to control an operation of each component of the computer 10. The CPU 111 executes various types of software loaded from the HDD 117 (or SSD) into the main memory 113. This software comprises an operating system (OS) 201 and various application programs. The application programs comprise a measurement program 202. The measurement program 202 can execute the processing of measuring the vital sign signal of the user in conjunction with the measurement engine 125.

The measurement engine 125 is configured to analyze the output time-series signal of the vital sign sensor, and to measure a value concerning the vital sign signal. The measurement engine 125 includes circuitry. The measurement engine 125 may comprise one or more processors and a memory storing a program executed by the one or more processors. Alternatively, the measurement engine 125 may be realized by dedicated hardware.

Further, the CPU 111 also executes a Basic Input/Output System (BIOS) stored in the BIOS-ROM 116 which is a non-volatile memory. The BIOS is a system program for hardware control.

The GPU 114 is a display controller which controls the LCD 31 used as a display monitor of the computer 10. The GPU 114 generates a display signal (LVDS signal) to be supplied to the LCD 31, from display data stored in a video memory (VRAM) 114A. Furthermore, the GPU 114 can generate an analog RGB signal and an HDMI video signal from display data. The analog RGB signal is supplied to an external display through the RGB port 24. The HDMI output terminal 23 can send out the HDMI video signal (incompressible digital video signal) and a digital audio signal to the external display by one cable. An HDMI control circuit 119 is an interface for sending out the HDMI video signal and the digital audio signal to the external display through the HDMI output terminal 23.

The system controller 112 is a bridge device which connects between the CPU 111 and each component. The system controller 112 incorporates a serial ATA controller for controlling the hard disk drive (HDD) 117 and the optical disk drive (ODD) 118. Furthermore, the system controller 112 communicates with each of devices on an LPC (low PIN count) bus.

The TV tuner 124 is configured to receive a TV broadcast signal and to perform channel selection. The EC/KBC 130 is connected to the LPC bus. The EC/KBC 130, the power-supply controller (PSC) 141, and the battery 20 are interconnected through a serial bus such as an I²C bus.

The EC/KBC 130 is a power management controller for executing power control of the computer 10, and is realized as a one-chip micro computer incorporating a keyboard controller which controls, for example, the keyboard (KB) 13 and the touchpad 14. The EC/KBC 130 comprises a function of powering on and powering off the computer 10 in accordance with an operation of the power switch 16 by the user. The control of powering on and powering off the computer 10 is executed by a cooperative operation between the EC/KBC 130 and the power-supply controller (PSC) 141. When receiving an on-signal transmitted from the EC/KBC 130, the power-supply controller (PSC) 141 controls the power-supply circuit 142 to power on the computer 10. Further, when receiving an off-signal transmitted from the EC/KBC 130, the power-supply controller (PSC) 141 controls the power-supply circuit 142 to power off the computer 10. The EC/KBC 130, the power-supply controller (PSC) 141, and the power-supply circuit 142 are operated by power from the battery 20 or an AC adaptor 150 also while the computer 10 is powered off.

Moreover, the EC/KBC 130 can turn on/off the keyboard backlight 13A arranged at the back of the keyboard 13. Furthermore, the EC/KBC 130 is connected to the panel opening and closing switch 131 configured to detect opening and closing of the display unit 12. The EC/KBC 130 also can power on the computer 10 when opening of the display unit 12 is detected by the panel opening and closing switch 131.

The power-supply circuit 142 generates power (operation power) to be supplied to each component using power from the battery 20, or power from the AC adaptor 150 connected to the computer main body 11 as external power-supply.

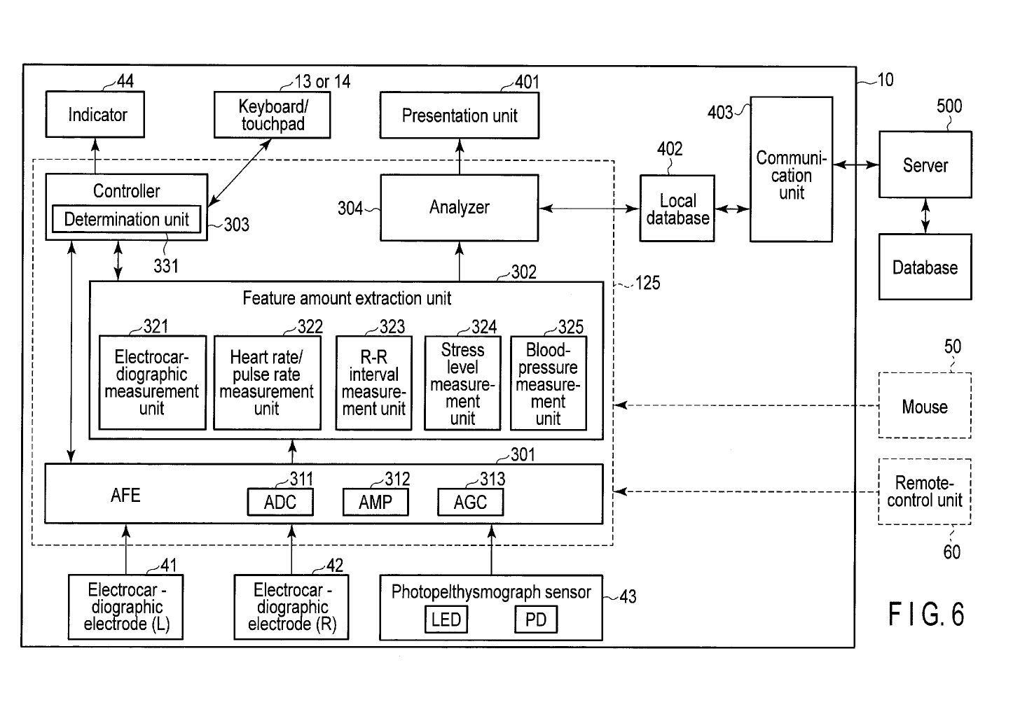

FIG. 6 shows a relationship between the measurement engine 125 provided in the computer 10 and components around the measurement engine 125.

The measurement engine 125 comprises analog front end (AFE) 301, a feature amount extraction unit 302, controller 303, and an analyzer 304. Analog front end 301 generates an output time-series signal corresponding to a detection signal of an electrocardiogram sensor by sampling an output signal of electrocardiogram sensor (a difference in potential between electrocardiographic electrodes 41 and 42). Further, analog front end 301 generates an output time-series signal corresponding to a detection signal of PPG sensor 43 by sampling an output signal of PPG sensor 43. Analog front end 301 is constituted of analog/digital converter (ADC) 311, amplifier (AMP) 312, automatic gain controller (AGC) 313, etc.

The feature amount extraction unit 302 functions as a measurement controller (circuitry) configured to analyze at least one of an output time-series signals of the electrocardiogram sensors (electrocardiographic electrodes 41 and 42) obtained by analog front end 301, and that of PPG sensor 43 obtained by analog front end 301, and to measure a value concerning a vital sign signal of a human body. The feature amount extraction unit 302 comprises an electrocardiographic measurement unit 321, a heart rate/pulse rate measurement unit 322, R-R interval measurement unit 323, stress level determination unit 324, and a blood-pressure measurement unit 325.

The electrocardiographic measurement unit 321 analyzes the output time-series signal of the electrocardiogram sensor, and measures the electrocardiogram. The heart rate/pulse rate measurement unit 322 executes processing of measuring a heart rate based on an electrocardiogram obtained by the electrocardiographic measurement unit 321, or processing of analyzing an output time-series signal of PPG sensor 43 to measure a pulse rate. R-R interval measurement unit 323 measures an R-R interval (RRI) which is an interval between two R waves corresponding to two successive heartbeats based on an electrocardiogram obtained by the electrocardiographic measurement unit 321.

Stress level measurement unit 324 analyzes the output time-series signal of PPG sensor 43, and generates the pulse interval data indicating variation of a pulse interval. Then, stress level measurement unit 324 measures a stress level based on a power spectrum (LF) of a low-frequency domain and a power spectrum (HF) of a high-frequency domain, each of which is obtained by converting pulse interval data for a predetermined time period into frequency spectrum distribution. In this case, LF/HF represents the stress level.

The blood-pressure measurement unit 325 measures the above-described pulse wave transit time (PWTT) based on the electrocardiogram and the pulse wave, and measures a blood pressure based on this PWTT and the above-described initial value, or based on the PWTT and the above-described normal data.

Controller 303 controls an operation of the measurement engine 125. In the present embodiment, controller 303 comprises determination unit 331 to allow the vital sign signal to be automatically measured while the user operates the keyboard 13, etc., of the computer 10. Determination unit 331 is a determination controller (circuitry) configured to determine whether the user (human body) is in contact with the vital sign sensor, and whether a contact state between the vital sign sensor and the user (human body) is stable, while the vital sign signal is detected by vital sign sensors (electrocardiographic electrodes 41 and 42, and PPG sensor 43).

Each of measurement units in the feature amount extraction unit 302 analyzes a time-series signal obtained by removing, from an output time-series signal obtained using the vital sign sensor, a time-series signal portion corresponding to a time period of a non-contact state where the user (human body) is not in contact with the vital sign sensor, and that corresponding to a time period of an unstable state where a contact state between the vital sign sensor and the user (human body) is unstable, and measures a value concerning a vital sign signal.

This allows the time-series signal portion corresponding to the time period of the non-contact state where a hand of the user is not in contact with the vital sign sensor, and that corresponding to the time period of the unstable state where the contact state is unstable to be automatically excluded from an object to be measured. Thus, the vital sign signal of the user can be automatically measured while the user operates the keyboard 13, etc., of the computer 10, even if a hand of the user is not still.

Further, in general, a detection signal (output time-series signal) for at least a specific time period is required in the measurement of the vital sign signal. In the present embodiment, each measurement unit analyzes a time-series signal for a specific time period which is obtained by connecting time-series signal portions corresponding to time periods of the stable state, and measures the vital sign signal.

For example, pulse wave data (output time-series signal concerning pulse wave) for a time period of approximately 20 seconds is required in the measurement of the stress level. In the present embodiment, stress level measurement unit 324 analyzes the time-series signal for the time period of approximately 20 seconds which is obtained by connecting time-series signal portions corresponding to the time periods of the stable state where the contact state between PPG sensor 43 and the user is stable, and measures the stress level. Thus, even if the user is not continuously still for 20 seconds with the user in contact with PPG sensor 43, the stress level can be measured when the total time of the time periods of the stable state reaches approximately 20 seconds.

Thus, the measurement of the vital sign signal can be performed without causing the user to be conscious of the measurement, or forcing a specific posture.

The measurement by each measurement unit in the feature amount extraction unit 302 may be regularly and repeatedly executed. A number of measurement results obtained by repeating regular measurements are accumulated in a local database 402 in the computer 10 by the analyzer 304. The analyzer 304 may calculate, for example, a weekly/monthly average value, and a weekly/monthly moving average value by statistically processing a number of measurement values accumulated in the local database 402. Further, the analyzer 304 may calculate change of yearly average values (secular change).

A presentation unit 401 presents a value concerning a vital sign signal obtained by the measurement, for example, a pulse, a blood pressure, and a stress level to the user. Weekly/monthly average values, weekly/monthly moving average values, etc., of a pulse, a blood pressure, and a stress level may be presented to the user.

Incidentally, guidance for notifying the user that the vital sign signal is sensed may be displayed to start measurement of the vital sign signal, when the computer 10 is powered on, and a login screen is displayed, or immediately after the computer 10 is powered on. To display the guidance, a screen for urging the user to put both hands on the palm rest area 40 may be displayed, a screen for urging the user to grasp the mouse 50 may be displayed, and a screen for teaching the user how to grasp the remote-control unit 60 may be displayed.

Further, a measurement value accumulated in the local database 402 may be transmitted to a server 500 by a communication unit 403.

Moreover, the measurement engine 125 can also receive a time-series signal from the vital sign sensor of the mouse 50 or that of the remote-control unit 60.

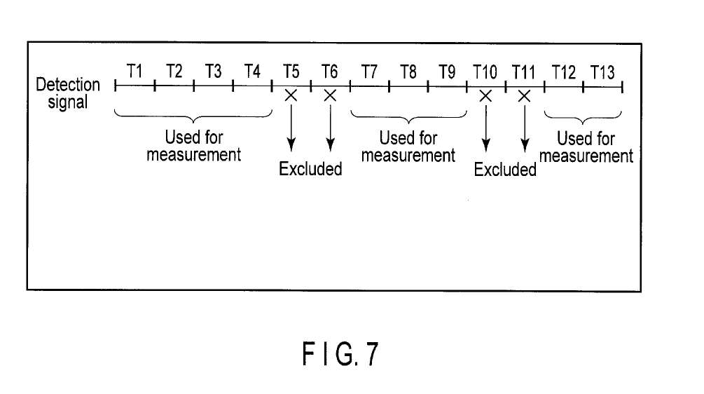

FIG. 7 illustrates an operation for removing a time-series signal portion in a time period other than that of a stable state from a detection signal (output time-series signal) of a vital sign sensor. The stable state refers to a state where a vital sign sensor and a human body are stably in contact with each other.

A case where it is determined that time periods T5, T6, T10 and T11 correspond to the non-contact state or the unstable state is hereinafter assumed. In this case, time-series signal portions corresponding to time periods T5 and T6 and those corresponding to time periods T10 and T11 are removed from signals (output time-series signals) of time periods T1 to T12. Then, the time-series signal portions of time periods T1 to T4, those of time periods T7 to T9, and those of time periods T11 and T12 are analyzed to measure the vital sign signal. Time-series signals for nine time periods are obtained by connecting the time-series signal portions of time periods T1 to T4, those of time periods T7 to T9, and those of time periods T11 and T12.

Determination unit 331 performs contact determination and stability determination for each of the output time-series signal of the electrocardiogram sensor and that of PPG sensor 43. The contact determination is an operation for determining whether the user (human body) is in contact with the vital sign sensor. The stability determination is an operation for determining whether the contact state between the vital sign sensor and the user (human body) is stable. In the stability determination, a state where the human body moves relative to the vital sign sensor is determined to be the unstable state where the contact state between the vital sign sensor and the user (human body) is not stable. This allows a time-series signal of a time period corresponding to a state where a hand, etc., of the user moves relative to the sensor to be excluded from an object to be analyzed.

Determination unit 331 can analyze a frequency characteristic of the time-series signal of the electrocardiogram sensor to determine whether a human body (skin) is in contact with electrocardiogram sensors (electrocardiographic electrodes 41 and 42) (contact determination), and to determine whether the contact state between the electrocardiogram sensors (electrocardiographic electrodes 41 and 42) and the human body (skin) are stable (stability determination).

More specifically, determination unit 331 can performs the contact determination and stability determination concerning the electrocardiogram sensor in the following manner.

A case where the sampling frequency of the output time-series signal of the electrocardiogram sensor is 1000 Hz is assumed.

<Contact Determination of Electrocardiogram Sensor>

Determination unit 331 determines that a time-series signal portion of the electrocardiogram sensor which does not comprise a frequency component (frequency component of 3 to 45 Hz) of a first frequency band is a time-series signal portion corresponding to a time period of a non-contact state where the user is not in contact with electrocardiogram sensors (electrocardiographic electrodes 41 and 42). Incidentally, the determination as to whether the user is in contact may be performed by measuring impedance of electrocardiographic electrodes 41 and 42 using hardware. Further, the determination as to whether the user is in contact may be performed using a proximity sensor.

<Stability Determination of Electrocardiogram Sensor>

Determination unit 331 determines that at least a time-series signal portion comprising whitened spectral distribution is a signal portion corresponding to a time period of an unstable state where a contact state between the electrocardiogram sensors (electrocardiographic electrodes 41 and 42) and the user is unstable. The time-series signal portion comprising the whitened spectral distribution (power spreads over the whole frequency) is frequently observed when a hand moves relative to electrocardiographic electrodes 41 and 42. Thus, the time-series signal portion comprising the whitened spectral distribution is preferably excluded from the object to be measured. Whether the whitened spectral distribution is included can be determined based on a spectral shape.

Moreover, determination unit 331 can also determine that a time-series signal portion whose power in a predetermined frequency band (3 to 12 Hz) is less than a predetermined value, as well as the time-series signal portion comprising the whitened spectral distribution, is a signal portion corresponding to a time period of the unstable state where the contact state where the electrocardiogram sensors (electrocardiographic electrodes 41 and 42) and the user is unstable.

Further, it is observed that the power in the predetermined frequency band (3 to 12 Hz) is reduced when the contact between electrocardiographic electrodes 41 and 42 and a hand is poor, that is, it is unstable. On the other hand, a harmonic structure from 1 to 30 Hz is observed when the contact between electrocardiographic electrodes 41 and 42 and a hand is stable. Thus, the signal portion whose power in the predetermined frequency band (3 to 12 Hz) is less than a predetermined value is preferably excluded from the object to be measured.

As described above, both of the contact determination and the stability determination are performed in the present embodiment, and then, the time-series signal portion comprising a frequency feature corresponding to the non-contact state and that comprising a frequency feature corresponding to the unstable state are specified. Then, each of the specified time-series signal portions is excluded from the object to be measured (analyzed). Accordingly, both of the signal portion corresponding to the time period of the non-contact state, and that corresponding to the time period of the unstable state where the contact state is unstable (hand moves relative to electrocardiographic electrodes 41 and 42, or contact is unstable) can be effectively removed.

Moreover, determination unit 331 can perform the contact determination and stability determination concerning pulse wave sensor 43 in the following manner.

A case where the sampling frequency of the output time-series signal of pulse wave sensor 43 is 125 Hz is hereinafter assumed.

<Contact Determination of Pulse Wave Sensor>

Determination unit 331 determines that a time-series signal portion of pulse wave sensor 43 which does not comprise a frequency component (frequency component of 5 to 50 Hz) of a first frequency band is a time-series signal portion corresponding to a time period of a non-contact state where the user is not in contact with pulse wave sensor 43. Incidentally, the determination as to whether the user is in contact can be determined using a proximity sensor.

<Stability Determination of Pulse Wave Sensor>

Determination unit 331 determines that a time-series signal portion comprising whitened spectral distribution, and a time-series signal portion in a predetermined frequency band (2 to 8 Hz) whose power is less than a predetermined value are a signal portion corresponding to a time period of an unstable state where a contact state between pulse wave sensor 43 and the user is unstable.

The time-series signal portion comprising the whitened spectral distribution (power spreads over the whole frequency) is frequently observed when a hand moves relative to pulse wave sensor 43. Thus, the time-series signal portion comprising the whitened spectral distribution is preferably excluded from the object to be measured. Whether the whitened spectral distribution is included can be determined based on a spectral shape.

Further, it is observed that the power in the predetermined frequency band (2 to 8 Hz) is reduced when the contact between pulse wave sensor 43 and a hand is poor, that is, it is unstable. Thus, the signal portion whose power in the predetermined frequency band (2 to 8 Hz) is less than a predetermined value is preferably excluded from the object to be measured.

As described above, both of the contact determination and the stability determination are performed on pulse wave sensor 43 in the present embodiment, and then, the time-series signal portion comprising a frequency feature corresponding to the non-contact state and that comprising a frequency feature corresponding to the unstable state are specified. Then, each of the specified time-series signal portions are excluded from the object to be measured (analyzed). Accordingly, both of the signal portion corresponding to the time period of the non-contact state, and that corresponding to the time period of the unstable state where the contact state is unstable (hand moves relative to pulse wave sensor 43, or contact is unstable) can be effectively removed.

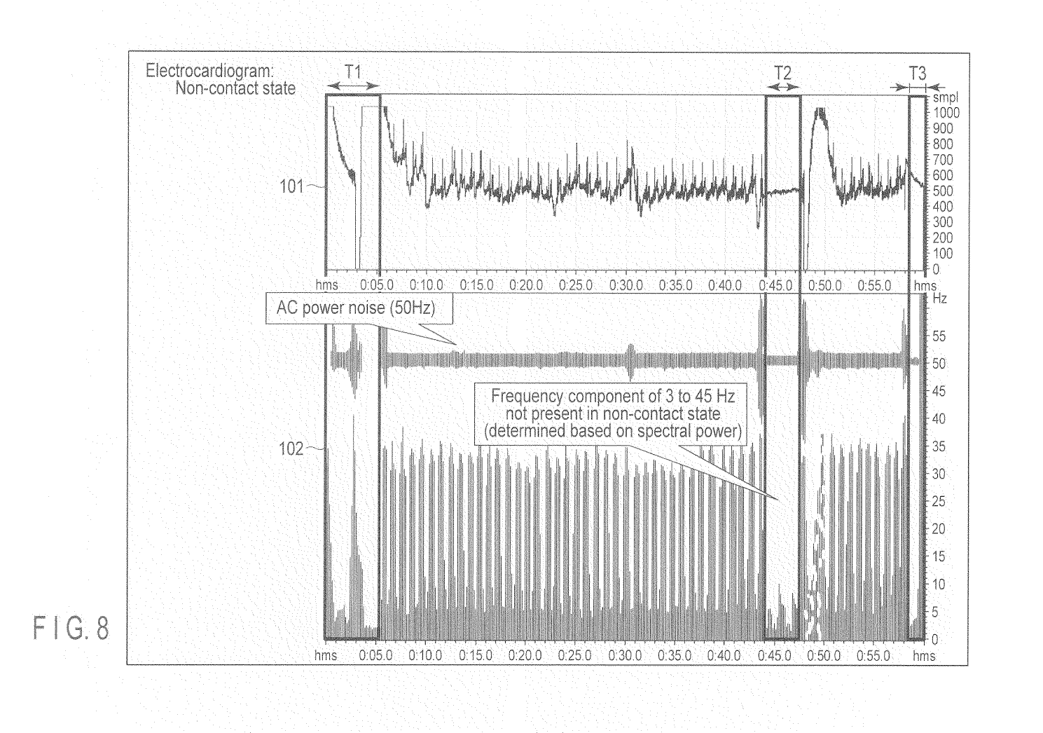

Next, referring to FIGS. 8-11, contact and stability determination operations to an output time-series signal of an electrocardiogram sensor will be described.

In FIGS. 8-11, graph 101 plots an output time-series signal (electrocardiogram signal) derived from the electrocardiogram sensor for approximately 60 seconds. The horizontal axis of graph 101 represents time (hms: hour/min/sec), and the vertical axis of graph 101 represents amplitude (smpl: sample). Graph 102 plots a frequency characteristic of the output time-series signal of the electrocardiogram sensor for 60 seconds. The horizontal axis of graph 102 represents time (hms: hour/min/sec), and the vertical axis of graph 102 represents frequency.

FIG. 8 is a view for illustrating a frequency characteristic of an electrocardiogram signal portion corresponding to a time period of a non-contact state. The frequency component of 3 to 45 Hz is not observed in the non-contact state. Thus, determination unit 331 determines that a time-series signal portion which does not comprise a frequency component of 3 to 45 Hz (that is, the time-series signal portion corresponding to time period T1, that corresponding to time period T2, and that corresponding to time period T3 in FIG. 8) is an electrocardiogram signal portion corresponding to the time period of the non-contact state. This allows the time-series signal portions of time periods T1 to T3 to be excluded from the electrocardiogram signal to be measured.

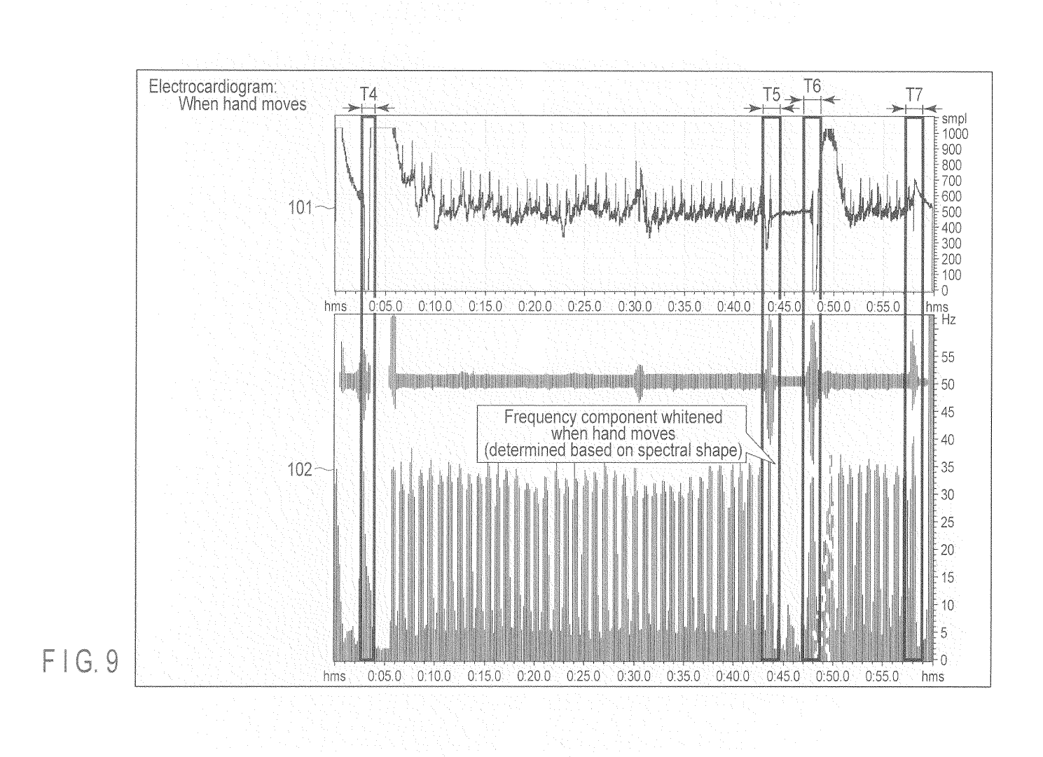

FIG. 9 is a view for illustrating a frequency characteristic of an electrocardiogram signal portion corresponding to a time period in which a hand moves relative to electrocardiogram sensors (electrocardiographic electrodes 41 and 42). A tendency of whitening of a frequency component (spectrum spreads over the whole frequency) is observed in the time period in which the hand moves. Thus, determination unit 331 determines that the time-series signal portion comprising the whitened spectral distribution (that is, the time-series signal portion corresponding to time period T4, that corresponding to time period T5, that corresponding to time period T6, and that corresponding to time period T7 in FIG. 9) is an electrocardiogram signal portion corresponding to the time period of the unstable state. This allows the time-series signal portions of time periods T4 to T7 to be excluded from the electrocardiogram signal to be measured.

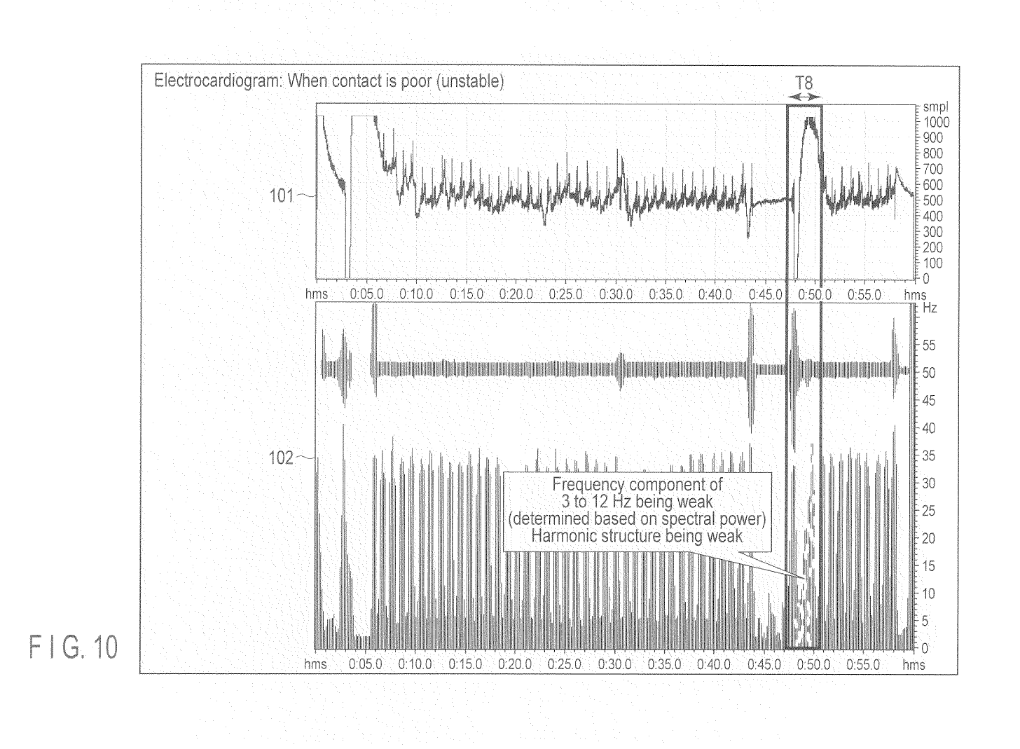

FIG. 10 is a view for illustrating a frequency characteristic of an electrocardiogram signal portion corresponding to a (unstable) time period in which contact is poor. A tendency for power of a frequency component of 3 to 12 Hz to be reduced is observed, and it is observed that a harmonic structure is also weak in the time period in which the contact is poor (unstable). Thus, determination unit 331 determines that the time-series signal portion whose power in the frequency band of 3 to 12 Hz is less than a predetermined value (that is, the time-series signal portion corresponding to time period T8 in FIG. 10) is an electrocardiogram signal portion corresponding to the time period of the unstable state. This allows the time-series signal portion of time period T8 to be excluded from the electrocardiogram signal to be measured.

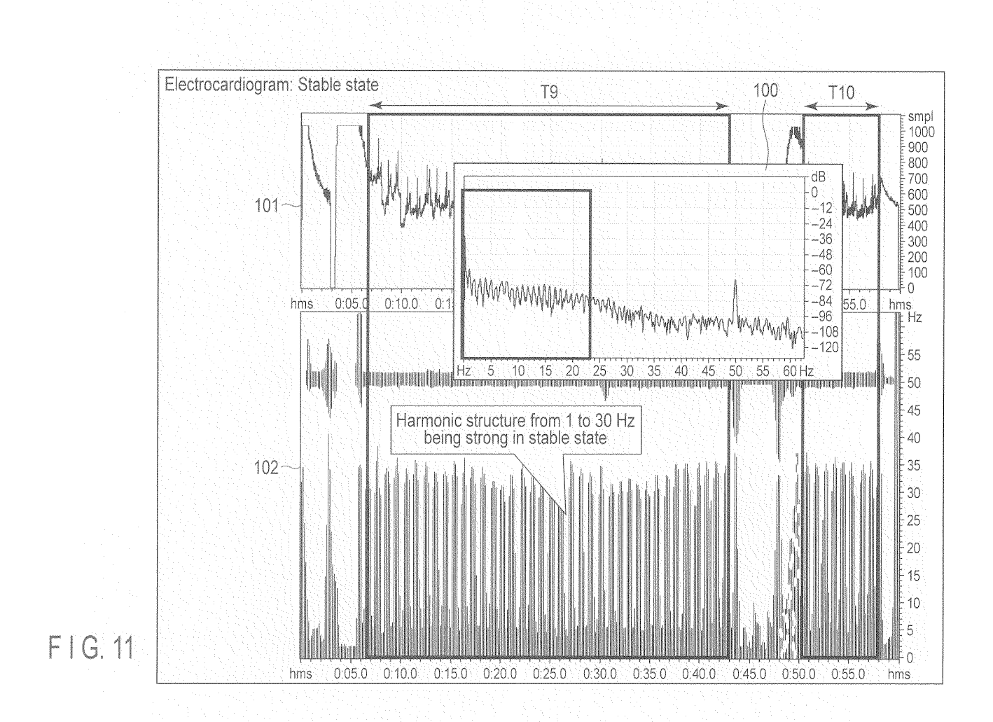

FIG. 11 is a view for illustrating a frequency characteristic of an electrocardiogram signal portion corresponding to a time period in which a contact state is stable. A strong harmonic structure is observed in the range of 1 to 30 Hz in the time period in which the contact state is stable. In FIG. 11, the time-series signal portion corresponding to time period T9 and that corresponding to time period T10 are electrocardiogram signal portions corresponding to the time period in which the contact state is stable. Graph 100 in FIG. 11 plots frequency distribution of the electrocardiogram signal portion corresponding to the time period in which the contact state is stable. It can be understood also from graph 100 that the electrocardiogram signal portion of the time period in which the contact state is stable comprises the strong harmonic structure in the range of 1 to 30 Hz.

Determination unit 331 can also specify the time-series signal portion comprising the strong harmonic structure in the range of 1 to 30 Hz as a time-series signal portion to be measured, instead of specifying the electrocardiogram signal portion corresponding to the time period of the unstable state to exclude the electrocardiogram signal portion corresponding to the time period of the unstable state from the object to be measured.

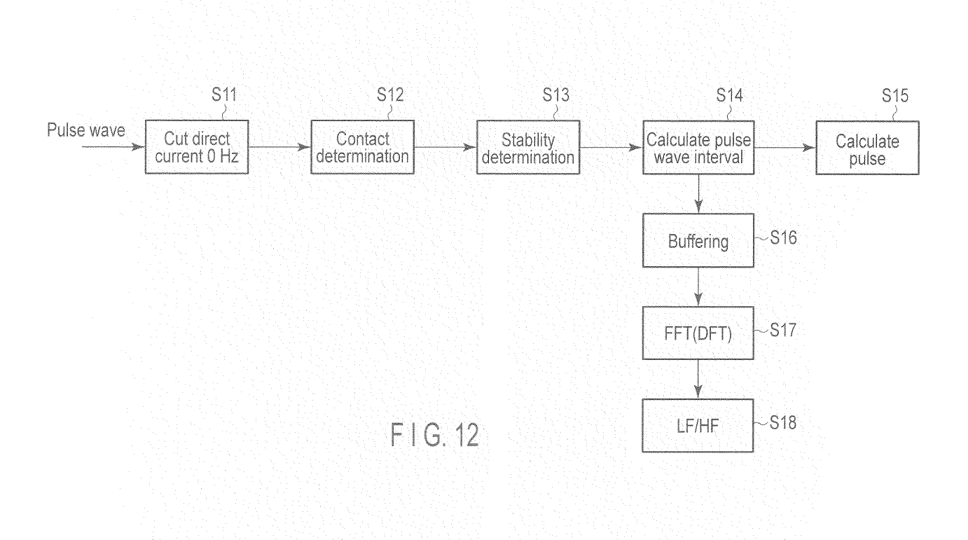

FIG. 12 is a view for illustrating processing of the output time-series signal (pulse wave signal) of pulse wave sensor 43.

The measurement engine 125 removes a direct-current component (noise) from a pulse wave signal using a high-pass filter, etc. (step S11). The measurement engine 125 determines whether a human body is in contact with pulse wave sensor 43 (step S12). In step S12, the measurement engine 125 can determine that a time-series signal portion which does not comprise a frequency component of 5 to 50 Hz is the time-series signal portion corresponding to the time period of the non-contact state. The measurement engine 125 determines whether a human body is stably in contact with pulse wave sensor 43, that is, whether the contact state between pulse wave sensor 43 and the human body is stable (step S13). In step S13, the measurement engine 125 can determine that the time-series signal portion comprising the whitened spectral distribution and the time-series signal portion whose power in 2 to 8 Hz is less than a predetermined value are the time-series signal portion of the time period of the unstable state where the contact state is not stable. Then, the measurement engine 125 calculates a pulse wave interval (step S14).

In step S14, the measurement engine 125 abandons the time-series signal portion corresponding to the time period of the non-contact state and that corresponding to the time period of the unstable state, and does not use the time-series signal portion corresponding to the time period of the non-contact state and that corresponding to the time period of the unstable contact state to calculate the pulse wave interval. In other words, the measurement engine 125 analyzes only a time-series signal portion corresponding to each time period of the stable state where the contact state is stable in real time to calculate the pulse wave interval. Then, a plurality of pulse wave interval data items, each of which indicates the pulse wave interval, are sequentially generated.

The measurement engine 125 calculates a pulse based on the generated pulse wave interval data items (step S15). Furthermore, the measurement engine 125 stores the generated pulse wave interval data items in a buffer (step S16). The measurement engine 125 executes frequency analysis of the plurality of pulse wave interval data items equivalent to the time period of approximately 20 seconds using fast Fourier transformation (FFT) or discrete Fourier transformation (DFT) (step S17). In step S17, every time one pulse wave interval data item is newly acquired, the oldest one of the pulse wave interval data items is abandoned. This causes the frequency analysis to be executed by the unit of a group of pulse wave interval data items which is equivalent to the time period of approximately 20 seconds. The above-described LF and HF are calculated by the frequency analysis. The measurement engine 125 calculates the LF/HF as a stress level (stress index) of the user (step S18).

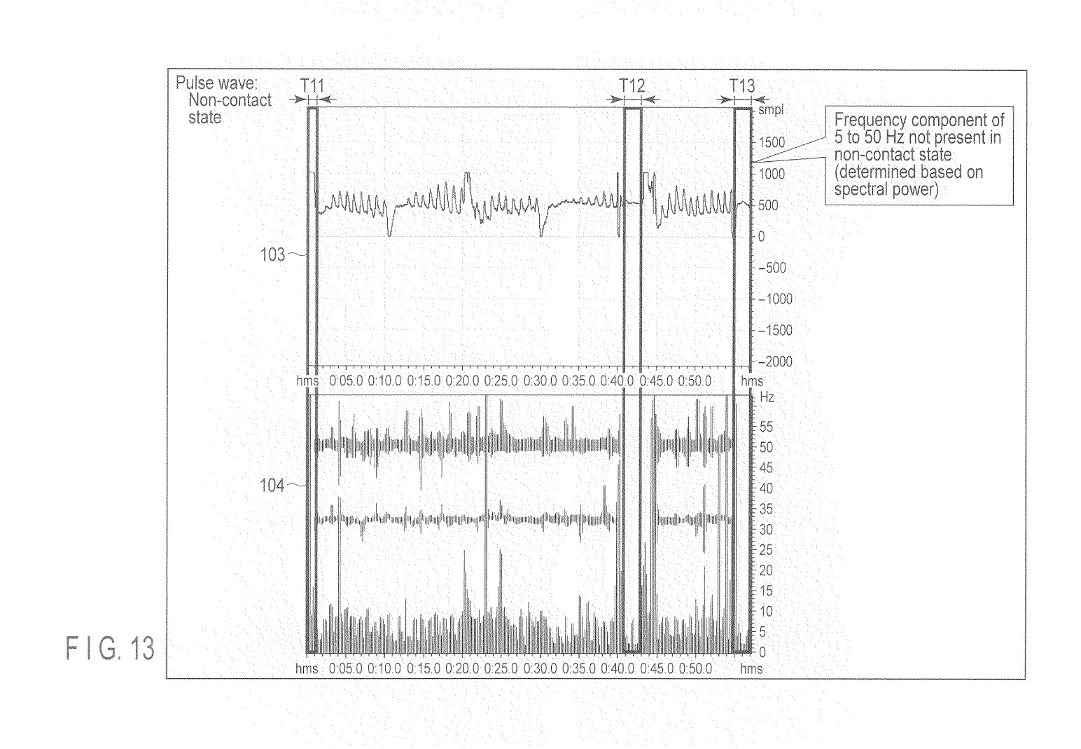

Next, referring to FIGS. 13-15, contact and stability determination operations to the output time-series signal of pulse wave sensor 43 will be described.

In FIGS. 13-15, graph 103 plots an output time-series signal (pulse wave signal) of pulse wave sensor 43 for approximately 60 seconds. The horizontal axis of graph 103 represents time (hms: hour/min/sec), and the vertical axis of graph 103 represents amplitude (smpl: sample). Graph 104 plots a frequency characteristic of the output time-series signal (pulse wave signal) derived from pulse wave sensor 43 for 60 seconds. The horizontal axis of graph 104 represents time (hms: hour/min/sec), and the vertical axis of graph 104 represents frequency.

FIG. 13 is a view for illustrating a frequency characteristic of a pulse wave signal portion corresponding to a time period of a non-contact state. The frequency component of 5 to 50 Hz is not observed in the non-contact state. Thus, determination unit 331 determines that a time-series signal portion which does not comprise a frequency component of 5 to 50 Hz (that is, the time-series signal portion corresponding to time period T12 and that corresponding to time period T13 in FIG. 13) is a pulse wave signal portion corresponding to the time period of the non-contact state. This allows the time-series signal portions of time periods T12 and T13 to be excluded from the pulse wave signal to be measured.

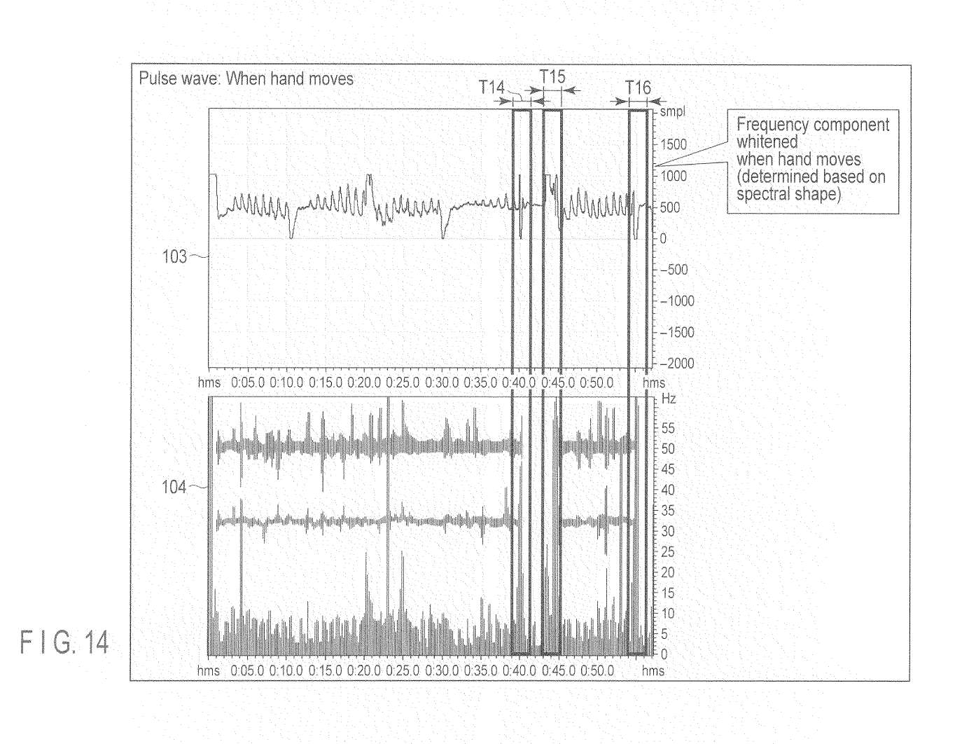

FIG. 14 is a view for illustrating a frequency characteristic of a pulse wave signal portion corresponding to a time period in which a hand moves relative to pulse wave sensor 43. A tendency of whitening of a frequency component (spectrum spreads over the whole frequency) is observed in the time period in which the hand moves. Thus, determination unit 331 determines that the time-series signal portion comprising the whitened spectral distribution (that is, the time-series signal portion corresponding to time period T14, that corresponding to time period T15, and that corresponding to time period T16 in FIG. 14) is a pulse wave signal portion corresponding to the time period of the unstable state. This allows the time-series signal portions of time periods T14 to T16 to be excluded from the pulse wave signal to be measured.

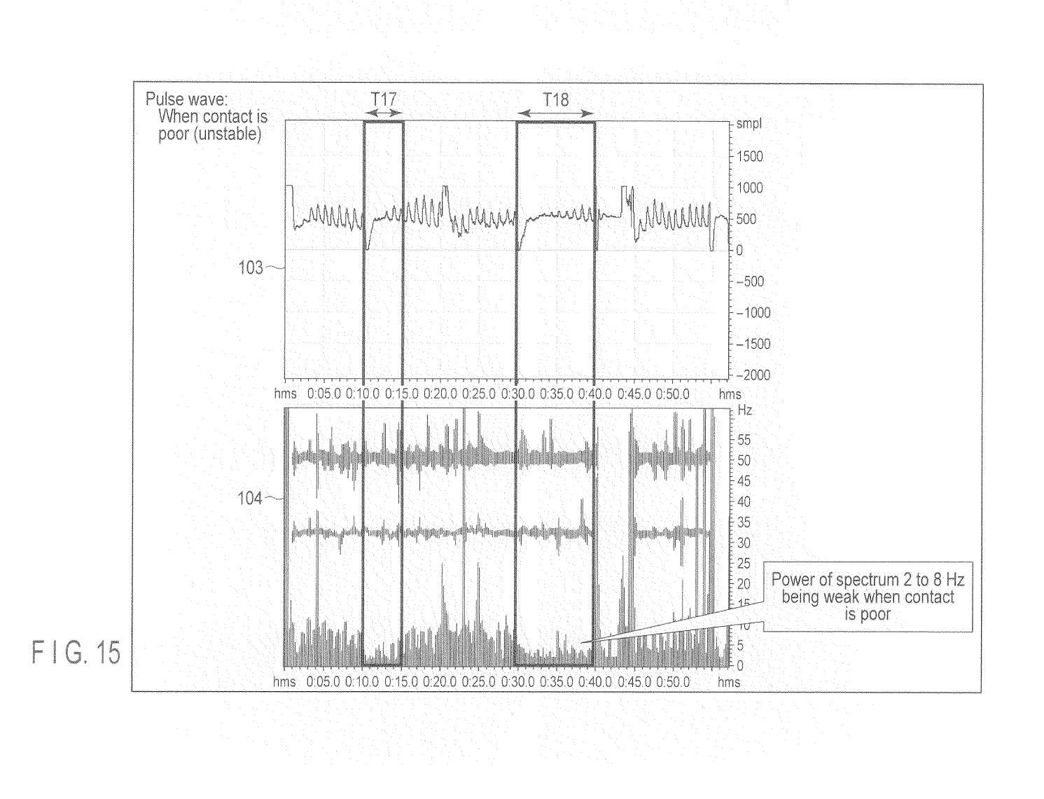

FIG. 15 is a view for illustrating a frequency characteristic of a pulse wave signal portion corresponding to a time period in which contact is poor (unstable). A tendency for power of a frequency component of 2 to 8 Hz to be reduced is observed in the time period in which the contact is poor (unstable). Thus, determination unit 331 determines that the time-series signal portion whose power in the frequency band of 2 to 8 Hz is less than a predetermined value (that is, the time-series signal portion corresponding to time period T17 and that corresponding to time period T18 in FIG. 15) is a pulse wave signal portion corresponding to the time period of the unstable state. This allows the time-series signal portions of time periods T17 and T18 to be excluded from the pulse wave signal to be measured.

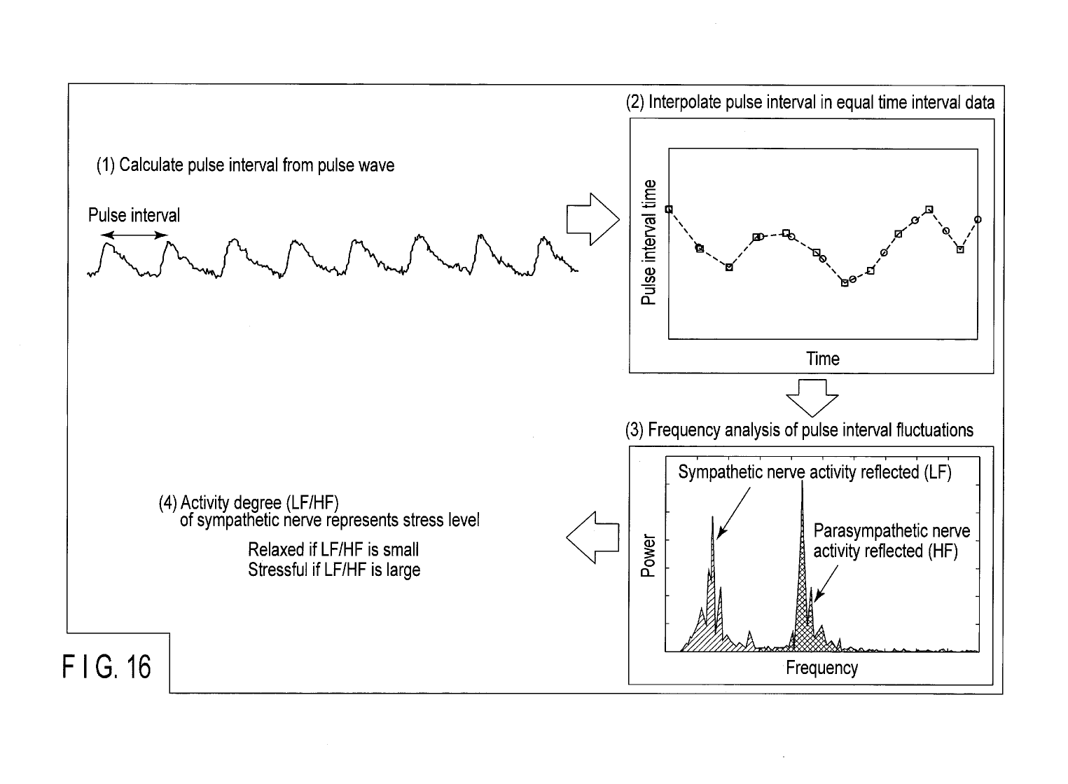

FIG. 16 is a view for illustrating an operation for calculating a stress level (stress index).

(1) Calculate Pulse Interval from Pulse Wave

The feature amount extraction unit 302 removes the time-series signal portion corresponding to the time period of the non-contact state and that corresponding to the time period of the unstable state from the output time-series signal of pulse wave sensor 43 to obtain a time-series signal (pulse wave signal) to be used to analyze a pulse interval. In other words, the feature amount extraction unit 302 connects time-series signal portions in an output time-series signal other than the time-series signal portion corresponding to the time period of the non-contact state and that corresponding to the time period of the unstable state (that is, time-series signal portions corresponding to each time period of the stable state) to obtain the time-series signal (pulse wave signal) to be used to analyze the pulse interval.

The feature amount extraction unit 302 detects a peak of each beat from the obtained pulse wave signal, and calculates a pulse interval indicating a time distance (pulse interval) between a detected peak and a peak immediately before the detected peak for each detected peak. Then, time-series pulse interval data indicating variation of the pulse interval is obtained.

(2) Interpolate Pulse Interval in Equal Time Interval Data

The feature amount extraction unit 302 interpolates time-series pulse interval data, and converts the time-series pulse interval data into equal time interval data (resampling). In the upper right graph of FIG. 16, a square indicates original pulse interval data, and a circle indicates pulse interval data obtained by interpolation.

(3) Frequency Analysis of Pulse Interval Fluctuations

The feature amount extraction unit 302 performs frequency analysis of equal time interval data, and calculates a power spectrum (LF) of a low-frequency domain, and a power spectrum (HF) of a high-frequency domain. The power spectrum (LF) of the low-frequency domain is a value reflecting sympathetic nerve activity, and the power spectrum (HF) of the high-frequency domain is a value reflecting parasympathetic nerve activity.

(4) Stress Level

The feature amount extraction unit 302 calculates activity degree (LF/HF) of a sympathetic nerve.

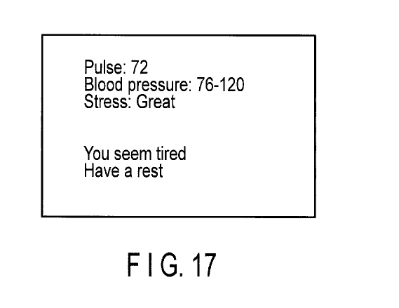

FIG. 17 shows an example of a measurement result presented to the user by the presentation unit 401.

The presentation unit 401 can display a pulse, a blood pressure, a stress level, etc., obtained by measurement on the screen of the LCD 31.

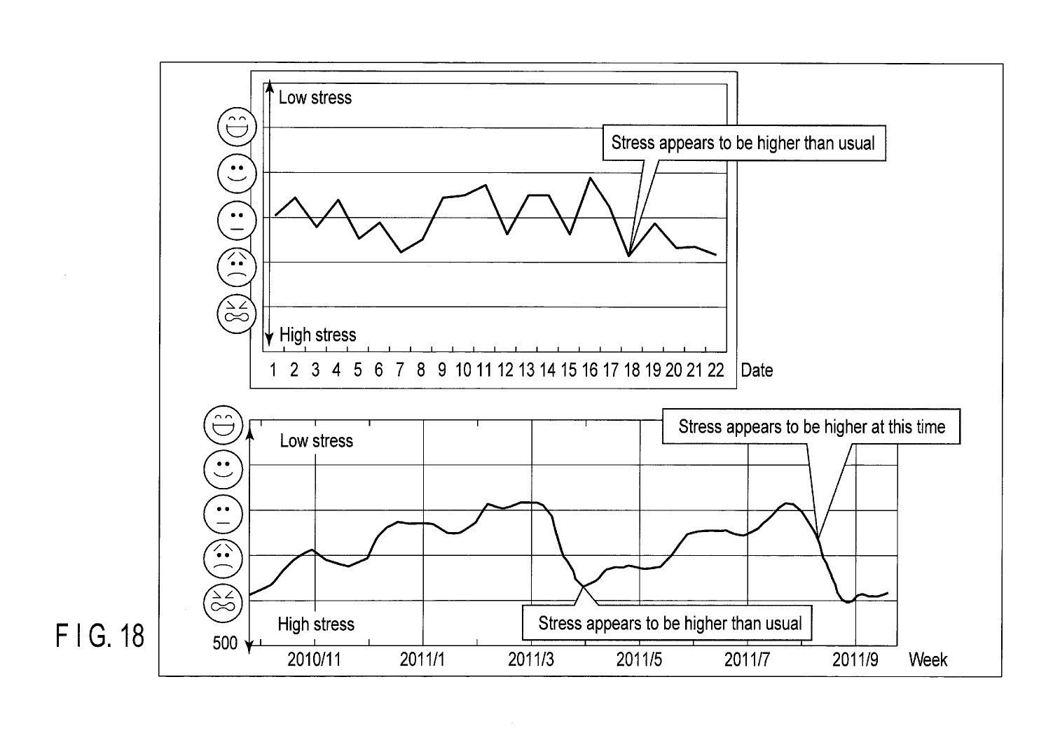

FIG. 18 shows another example of a measurement result presented to the user by the presentation unit 401.

The analyzer 304 calculates moving average, etc., of a stress level of the user using statistical information stored in the local database 402 (plurality of stress level measurement results). Then, the presentation unit 401 displays a graph indicating variation of the stress level of the user by the unit of a day or a week on the screen of the LCD 31. The graph shown in the upper portion of FIG. 18 is a line graph indicating the variation of the stress level by the unit of a day. A message “stress appears to be higher than usual” may be displayed at a position on the line graph corresponding to a day with high stress level. The graph shown in the lower portion of FIG. 18 is a line graph indicating the variation of the stress level by the unit of a week.

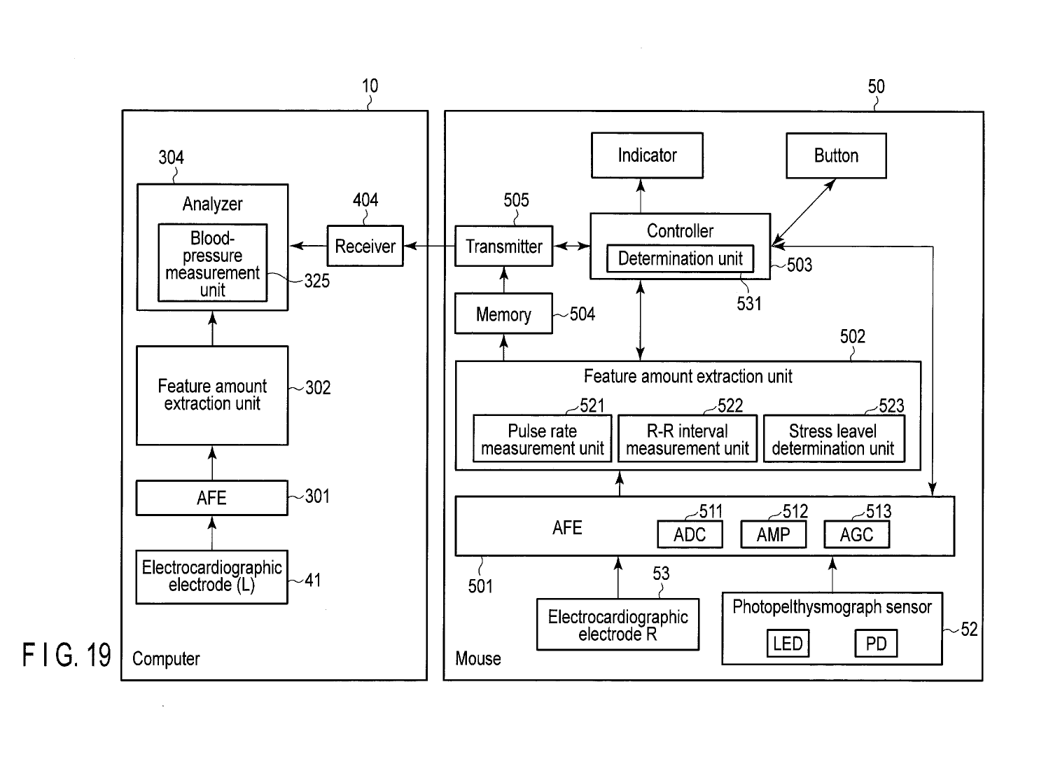

FIG. 19 shows a collaborative operation between the computer 10 and the mouse 50.

The mouse 50 comprises analog front end 501, a feature amount extraction unit 502, controller 503, a memory 504, and a transmitter 505, etc, as well as PPG sensor 52 and electrocardiographic electrode 53 which are described above.

Analog front end 501 generates an output time-series signal corresponding to a detection signal of PPG sensor 52 by sampling an output signal of PPG sensor 52. Further, analog front end 501 also generates an output time-series signal corresponding to electrocardiographic electrode 53 by sampling an electrical potential of electrocardiographic electrode 53. Analog front end 301 is constituted of analog/digital converter (ADC) 511, amplifier (AMP) 512, automatic gain controller (AGC) 513, etc.

The feature amount extraction unit 502 functions as a measurement controller configured to analyze an output time-series signal of PPG sensor 52 obtained by analog front end 501, and to measure a value concerning a vital sign signal of a human body. The feature amount extraction unit 502 comprises a pulse rate measurement unit 521, R-R interval measurement unit 522, and stress level measurement unit 523. The pulse rate measurement unit 521 analyzes the output time-series signal of PPG sensor 52, and measures a pulse rate. R-R interval measurement unit 522 analyzes the output time-series signal of PPG sensor 52, and measures an R-R interval (or pulse wave interval). Stress level measurement unit 523 analyzes the output time-series signal of PPG sensor 52, and measures a stress level, as with stress level measurement unit 324 in the computer 10.

Determination unit 503 in controller 503 is a determination controller configured to perform contact determination and stability determination on the output time-series signal of PPG sensor 52 by a procedure similar to that of determination unit 331 in the computer 10. Each of the pulse rate measurement unit 521, R-R interval measurement unit 522, and the stress level determination unit 523 analyzes a time-series signal obtained by connecting time-series signal portions corresponding to each time period of the stable state where the contact state between PPG sensor 52 and a human body is stable.

The mouse 50 may comprise an indicator such as an LED. In this case, controller 503 can notify the user, for example, that the vital sign signal is being measured by blinking, etc., of the indicator.

A measurement result obtained by the feature amount extraction unit 502 and an output time-series signal corresponding to electrocardiographic electrode 53 are stored in the memory 504. The transmitter 505 extracts the measurement result and an output time-series signal of electrocardiographic electrode 53 from the memory 504, and transmits the measurement result and the output time-series signal to the computer 10 through PS/S, USB, a Bluetooth module, or the like. The measurement result and the output time-series signal of electrocardiographic electrode 53 may be stored in the local database 402 in the computer 10.

The computer 10 can measure the electrocardiogram using the output time-series signal obtained by sampling the electrical potential of electrocardiographic electrode 41, and the output time-series signal of electrocardiographic electrode 53 received from the mouse 50 by a receiver 404.

Moreover, the receiver 404 can also receive an output time-series signal of a pulse wave from the mouse 50. In this case, the computer 10 can measure a blood pressure using the electrocardiogram and the output time-series signal of the pulse wave received from the mouse 50. In FIG. 19, a case where the analyzer 304 in the computer 10 comprises the blood-pressure measurement unit 325 configured to measure the blood pressure is shown as an example.

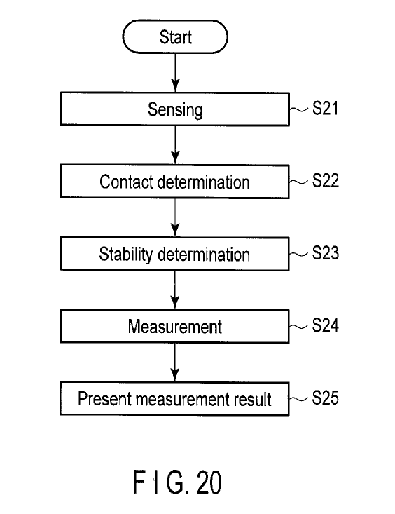

The flowchart in FIG. 20 shows a procedure of vital sign signal measuring processing executed by the measurement engine 125.

The measurement engine 125 measures (senses) a vital sign signal using vital sign sensors (PPG sensor 43 and electrocardiographic electrodes 41 and 42) (step S21). While it is sensed, the measurement engine 125 performs the contact determination, and determines whether a human body (skin) is in contact with the vital sign sensor (step S22). While it is sensed, the measurement engine 125 also performs the stability determination, and determines whether the contact state between the vital sign sensor and the human body (skin) is stable (step S23).

Then, the measurement engine 125 deletes the time-series signal portion corresponding to the time period of the non-contact state and that corresponding to the time period of the unstable state from the output time-series signal of the vital sign sensor. Thus, the time-series signal to be analyzed which is obtained by connecting time-series signal portions corresponding to each time period of the stable state is generated. The measurement engine 125 analyzes the time-series signal to be analyzed to measure a value concerning the vital sign signal (step S24), and presents this measurement result to the user (step S25).

As has been described above, according to the present embodiment, the contact determination and the stability determination are performed, and a time-series signal obtained by removing, from the output time-series signal of the vital sign sensor, a first time-series signal portion of the time period corresponding to the non-contact state, and a second time-series signal portion corresponding to the time period of the unstable state is analyzed. Thus, the measurement of the vital sign signal can be performed without causing the user to be conscious of the measurement, or forcing a specific posture.

Further, the time-series signal to be analyzed is obtained by connecting time-series signal portions in the output time-series signal other than the first time-series signal portion and the second time-series signal portion. Thus, if the total time in which the user is in a resting state (corresponding to the contact stable state) reaches a predetermined time, the time-series signal for a predetermined time which is required for the measurement can be obtained even if the user is not still for a long time. Accordingly, the vital sign signal can be measured while the user works using the computer 10.

Incidentally, the remote-control unit 60 in FIG. 4 may be a remote-control unit for remotely controlling a TV. In this case, the TV may comprise a function of the measurement engine 125. The TV can measure a value concerning a vital sign signal of the user while the user views or operates the TV.

Further, a structure in which the processing of measuring the value concerning the vital sign signal of the user is executed by an external server may be adopted, instead of a structure in which the processing of measuring the value concerning the vital sign signal of the user is performed in the computer 10 or the TV. In this case, for example, the computer 10 or the TV may transmit to a server a time-series signal obtained by removing, from an output time-series signal of the vital sign sensor, a first time-series signal portion corresponding to a time period of a non-contact state and a second time-series signal portion corresponding to a time period of an unstable state.

Further, since the processing procedure of the present embodiment can be executed by a computer program, an advantage similar to that of the present embodiment can be easily achieved merely by installing the computer program in a computer through a computer-readable storage medium storing the computer program, and executing the computer program.

The present embodiment is not limited to the above, but may be modified in various ways without departing from the scope. Various embodiments can be realized by appropriately combining the structural elements disclosed in the embodiments. For instance, some of the disclosed structural elements may be deleted. Some structural elements of different embodiments may be combined appropriately.

What is claimed is:

1. An electronic apparatus comprising:
a determination controller configured to determine whether a vital sign sensor is in contact with a human body and to determine whether a contact state between the vital sign sensor and the human body is stable; and
a measurement controller configured to:
obtain an effective time-series signal by removing, from an output time-series signal of the vital sign sensor, a first time-series signal and a second time-series signal, the first time-series signal corresponding to a time period of a non-contact state where the vital sign sensor is not in contact with the human body and a second time-series signal corresponding to a time period of an unstable state where a contact state between the vital sign sensor and the human body is unstable; and
analyze the effective time-series signal to measure a value associated with a vital sign signal of the human body.

2. The electronic apparatus of claim 1, wherein
the effective time-series signal is obtained by connecting time-series signal portions in the output time-series signal other than the first time-series signal and the second time-series signal.

3. The electronic apparatus of claim 1, wherein
the unstable state comprises a state where the human body moves relative to the vital sign sensor.

4. The electronic apparatus of claim 1, wherein
the determination controller is configured to determine that a time-series signal portion is the second time-series signal when the output time-series signal comprises a white spectral distribution.

5. The electronic apparatus of claim 1, wherein
the determination controller is configured to determine that a time-series signal portion is the second time-series signal when the output time-series signal comprises a white spectral distribution, and/or power of the time-series signal portion less than a first value in a predetermined frequency band.

6. The electronic apparatus of claim 1, wherein
the determination controller is configured to:
determine that a time-series signal portion in the output time-series signal is the first time-series signal when the time-series portion does not contain a frequency component in a first frequency band; and
determine that a time-series signal portion in the output time-series signal is the second time-series signal when the time-series portion comprises a white spectral distribution, and/or power of the time-series signal portion less than a first value in a predetermined frequency band.

7. The electronic apparatus of claim 1, wherein
the vital sign sensor comprises a pulse wave sensor, and
the measurement controller is configured to:
generate pulse interval data indicative of variations of pulse intervals, based on the effective time-series signal obtained by removing the first time-series signal and the second time-series signal from an output time-series signal of the pulse wave sensor; and
measure a stress level based on a low-frequency power spectrum and a high-frequency power spectrum of frequency spectrum distribution, the frequency spectrum distribution is converted from the pulse interval data for a predetermined time period.

8. The electronic apparatus of claim 1, wherein
the electronic apparatus is configured to process information received from an input device, and
the vital sign sensor is on a part of a housing of the electronic apparatus which a hand contacts when the input device is operated, or in the input device.

9. The electronic apparatus of claim 1, further comprising:
a main body comprising a keyboard on an upper surface; and
a display attached to the main body, and configured to display a value of the vital sign signal,
wherein the vital sign sensor is in a palm rest area on the upper surface.

10. The electronic apparatus of claim 1, wherein
the vital sign sensor is in a mouse configured to communicate with the electronic apparatus.

11. The electronic apparatus of claim 1, wherein
the vital sign sensor is in a remote-control unit configured to communicate with the electronic apparatus.

12. The electronic apparatus of claim 9, wherein
the vital sign sensor comprises a first and a second electrocardiographic electrode on both sides of a touchpad on the palm rest area.

13. The electronic apparatus of claim 9, wherein
the vital sign sensor comprises a pulse wave sensor on the palm rest area.

14. The electronic apparatus of claim 9, wherein
the vital sign sensor comprises a first and a second electrocardiographic electrode on both sides of a touchpad on the palm rest area, and a pulse wave sensor in proximity to the first electrocardiographic electrode or the second electrocardiographic electrode.

15. The electronic apparatus of claim 9, wherein
the vital sign sensor comprises a first and a second electrocardiographic electrode plate on both sides of a touchpad on the palm rest area, and a pulse wave sensor arranged to be exposed through an opening on the first electrocardiographic electrode plate or the second electrocardiographic electrode plate.

16. The electronic apparatus of claim 1, further comprising:
a main body comprising a key board on an upper surface; and
a display attached to the main body, and configured to display a value of the vital sign signal,
wherein the vital sign sensor comprises a first electrocardiographic electrode in a palm rest area on the upper surface, and a second electrocardiographic electrode provided in a mouse configured to communicate with the electronic apparatus.

17. A method for measuring a vital sign signal, the method comprising:
determining whether a vital sign sensor is in contact with a human body;
determining whether a contact state between the vital sign sensor and the human body is stable;
obtaining an effective time-series signal by removing, from an output time-series signal of the vital sign sensor, a first time-series signal and a second time-series signal, the first time-series signal corresponding to a time period of a non-contact state where the vital sign sensor is not in contact with the human body and the second time-series signal corresponding to a time period of an unstable state where a contact state between the vital sign sensor and the human body is unstable; and
analyzing the effective time-series signal to measure a value associated with a vital sign signal of the human body.

18. A computer-readable, non-transitory storage medium having stored thereon a computer program which is executable by a computer, the computer program controlling the computer to execute functions of:
determining whether a vital sign sensor is in contact with a human body;
determining whether a contact state between the vital sign sensor and the human body is stable;
obtaining an effective time-series signal by removing, from an output time-series signal of the vital sign sensor, a first time-series signal and a second time-series signal, the first time-series signal corresponding to a time period of a non-contact state where the vital sign sensor is not in contact with the human body and the second time-series signal corresponding to a time period of an unstable state where a contact state between the vital sign sensor and the human body is unstable; and
analyzing the time-series signal to measure a value associated with a vital sign signal of the human body.
